(19) United States
(12) Patent Application Publication
(10) Pub. No.: US 2017/0296316 A1
(43) Pub. Date: Oct. 19, 2017

(54) METHOD FOR ENHANCED REMINERALIZATION OF TEETH

(71) Applicant: DBD Innovations Pty Ltd, Helensburgh (AU)

(72) Inventors: Piotr Glowacki, Umina Beach (AU); Alexander Ian McIntosh, North Turramurra (AU); Guy James Reynolds, Helesburgh (AU)

(73) Assignee: DBD Innovations Pty Ltd

(21) Appl. No.: 15/130,969

(22) Filed: Apr. 16, 2016

(51) Int. Cl. A61C 19/06; H05H 1/24; A61N 1/44

(52) U.S. Cl. CPC A61C 19/06; A61N 1/44; H05H 1/2406

(57) ABSTRACT

A method for the treatment of teeth which comprises enhancing transport of substances through the tooth enamel by generating a gaseous plasma in proximity to the tooth.

### RELATED PRIOR ART

The invention refers to the own prior art of the Inventors, as disclosed in U.S. patent application Ser. No. 14/474,060 published on 2015 Apr. 23, publication number 2015/0112300.

### TECHNICAL FIELD

The present invention relates to a method of remineralization of teeth using a dielectric barrier discharge.

### BACKGROUND OF THE INVENTION

The invention takes advantage of known properties of Dielectric Barrier Discharge Discharges (DBD) and Iontophoresis.

Iontophoresis, which is also known as Electromotive Drug Administration, is a well-researched technique for delivering therapeutic substances using a small electric charge to transport the substances through a human or animal tissue under the action of an applied electric field.

Iontophoresis is used as a non-invasive method of delivering a medication or other bioactive agent into the body. In medicine, Iontophoresis is typically used trans-dermally. In dentistry, Iontophoresis can be used to facilitate transport of substances across the oral mucosa, tooth enamel, dentin or cementum.

Iontophoresis relies on active transportation within an electric field, where electro- migration and electro-osmosis are the dominant forces in mass transport.

In conventional Iontophoresis, an electrical power supply is used to apply a constant (DC) or alternating (AC) voltage, with or without a DC voltage bias, between the tissue and the chemical vessel, creating an ion flux.

DBD is the electrical discharge between two electrodes separated by an insulating dielectric barrier(s) while the electrodes are subjected to a high, alternating voltage. It has found numerous applications in science, industry, and medicine due to operating at strongly non-equilibrium conditions in various gases, including air, at atmospheric pressure, at reasonably high power levels while not necessarily using sophisticated power supplies or circuitry.

DBD plasma is typically obtained when the electrodes are separated by a gap of some millimetres and excited by alternating high voltage of frequency typically in the range 1 Hz-10 MHz. A DBD in air at atmospheric pressure can be created using a voltage as low as about 300V for a distance between the electrodes of about 10 micrometers. Typically, the DBD in air is formed by a large number of separate current filaments referred to as micro-discharges. The micro-discharges have a typical diameter in the range of 100 micrometers. These micro-discharges have a complex dynamic structure and are formed by channel streamers that usually repeatedly strike at the same place as the polarity of the applied voltage changes, thus appearing as bright filaments.

In certain cases, though, such as when the applied high voltage rise time is extremely short (for example dV/dt>1kV/ns), the microfilaments may not form any stable pattern and the discharge may appear uniform.

The micro-streamers are, however, extremely short lived. The avalanche to streamer transition generally takes about 10 ns, followed by the extinction of the micro-streamers. The extinction voltage of the micro-discharges is not far below the voltage of their ignition. Charge accumulation on the surface of the dielectric barrier reduces the electric field at the location of a micro-discharge, which results in current termination within tens of nanoseconds after breakdown. The short duration of current in the micro-discharges leads to low heat dissipation and, as such, the DBD plasma remains substantially non-thermal.

Importantly, the DBD is non-destructive as there is no significant rise of gas temperature and no electrical arc generation between the electrodes. Since the human or animal body is predominantly made of water, the tissue dielectric constant is sufficiently high to create DBD between the device electrode and the tissue. Most of the applied voltage appears across the DBD gap between the electrode and the body but the electrical current is held at safe level. Such an arrangement is usually described as Floating Electrode Dielectric Barrier Discharge (FEDBD), as because a ground connection is not necessary. The low temperature, low current and non-destructive nature of the discharge renders this methodology safe for use on patients.

As a result of the phenomena described in [0011] and [0012] above, a DBD can be safely applied to living tissues. In this case, the tissue acts as an electrode while high, alternating voltage is applied to the device electrode which is covered with dielectric. A DBD is created in air between the tissue and the device electrode.

The application of a DBD to human tissue in order to sterilize the tissue, coagulate blood, kill cancerous cells and the like has been extensively researched and published, in particular by Zemel et al in US 2013/0345620. However, the application of a DBD specifically in order to promote the transport of substances has not previously been taught or implied except in our own prior art referred to in [0001].

Importantly, due to the high voltage and high frequency nature of the DBD discharge, its application augments the mobility of molecules, both charged and neutral, increasing the efficiency of the delivery of the desired species through the tissue. Consequently, the method described herewith offers advantages over conventional Iontophoresis, which only facilitates the transport of ions.

Ions move together with the surrounding water molecules when they are hydrated. The effective Stokes radius of an ion is estimated to be half of the whole size of the ion attached to the hydrating water molecule/s. When such ion vibrates due to the DBD induced alternating electric field, the interactions between the ion and water molecules are affected, resulting in a reduction in the effective Stokes radius. This leads to an increase in the diffusion efficiency.

This process affects all hydrolyzed molecules, both electrically charged and neutral, increasing the efficiency of diffusion-based transport of the molecules.

In case where the electrodes are of different area, shape or material, a DC bias voltage may develop between the electrodes due to different mobility of electrons and ions, and different rate of absorption and removal of electrons and ions from electrodes of different materials.

In the particular case of a plasma in air between the tooth enamel and the dielectrically coated electrode, such a plasma-induced DC electric field will further enhance the transport of ions through the tooth enamel.

DBD enhanced process may be combined with a DC or AC based method. For example, a DC bias could be applied while the DBD is in operation to promote diffusion of specific ion species. Furthermore, as the DBD duty cycle is usually low, a DC bias could be introduced between the DBD impulses to similar effect. Moreover, the rest period between pulses may be beneficial, allowing completion of mass transport initiated by the applied voltage.

Furthermore, the waveform of the signal used to generate the DBD can be asymmetrical. For example, it could consist of very short, high-voltage pulses superimposed on direct (DC) or alternating (AC) bias voltage signal of arbitrary shape. Such superimposition may produce an electrical field of non-zero average value to promote transport of specific ion species.

Furthermore, the direct (DC) or alternating (AC) bias voltage can be applied using an auxiliary electrode which is substantially electrically separated from the DBD electrode. Such auxiliary electrode can be touching the tooth directly, or be separated from the tooth. The auxiliary electrode could be placed remotely from the DBD electrode, for example within the handheld part of the chassis of the device to provide electrical bias or ground connection to the patient's body, or in close proximity to the DBD electrode, for example around the DBD electrode or within the DBD electrode area of the device.

Such auxiliary electrode may be used to limit or expand the size of the treated area. The electrical field or current created by the auxiliary electrode could inhibit or promote transport of specific ions on the surface of the enamel or within the enamel.

The DBD discharge will ionize the molecules of the chemicals on or adjacent to the surface of the tooth enamel, increasing the concentration of desired species of the ions on and near the surface of the enamel. This will create a concentration gradient of the ions, further promoting their diffusion into the enamel.

Tooth enamel, dentine and cementum undergo continual chemical and physical changes, which include loss of substances, which are essential for maintaining the enamel, dentine and cementum integrity, e.g. Fluoride, Calcium or Phosphate. A variety of compounds may be used to help replenish these substances to the necessary levels. See Wang patent, US publication 1990/4969868.

If the teeth are coated with suitable mixtures or compounds containing one or more of Fluoride, Calcium and Phosphate ions, the applied DBD drives the transport of these ions into sub-surface layers of the tooth where, through the process of remineralization, the tooth structure is repaired and strengthened, thereby helping to prevent, and potentially even reverse, the early stages of tooth decay

### BRIEF SUMMARY OF THE INVENTION

The present invention seeks to take advantage of the properties of a Dielectric Barrier Discharge specifically to enhance the transport rate and penetration depth of both ions and electrically neutral molecules through the tooth enamel, thereby making remineralization of the tooth enamel, dentine and cementum more versatile, more efficient and faster than other methods known in the art. Importantly, the present method allows remineralization to take place at greater depths below the surface of the tooth than anything known in the prior art and at depths where tooth demineralization, the known precursor of tooth decay, typically takes place and where remineralization of the tooth is most needed.

In accordance with the present invention, there is provided a method of remineralization of tooth enamel, including:

a. applying the remineralizing substances to a tooth, e.g. by covering the tooth with material (liquid, solid, gas, spray, paste, foam, gel, or vapor) of sufficient concentration of fluoride, calcium or phosphate;

b. placing a dielectric coated electrode in proximity to the tooth;

c. applying a voltage signal to generate a Dielectric Barrier Discharge (DBD) between the tooth and the electrode;

d. optionally moving the electrode relative to the surface of the tooth;

e. optionally applying an auxiliary DC or AC voltage bias between the tooth and the electrode to further enhance the transport of desired species and, subsequently, the remineralization of the tooth substance.

Preferably, the method includes providing the electrode as a portable dielectric electrode.

DBD enhance remineralization of teeth has the following advantages compared with previous iontophoretic methods known in the art;

a. increased rate of transport and depth of penetration of the desired molecules, both electrically charged and neutral, into the tooth substances, due to enhanced diffusion rate resulting from increased kinetic energy, increased gradient of ionized molecules and/or the reduction in Stokes radius of hydrolyzed species;

b. increased ionization of molecules;

c. sterilization of the tooth;

### BRIEF DESCRIPTION OF THE DRAWINGS

The invention is more fully described, by way of non-limiting example only, with reference to the accompanying drawings, in which:

FIG. 1 is a substitute circuit diagram of DBD application to a tooth;

FIG. 2 is a circuit diagram for generation of DBD;

FIG. 3 is a diagrammatic representation illustrating the method of the invention;

### DETAILED DESCRIPTION OF THE INVENTION

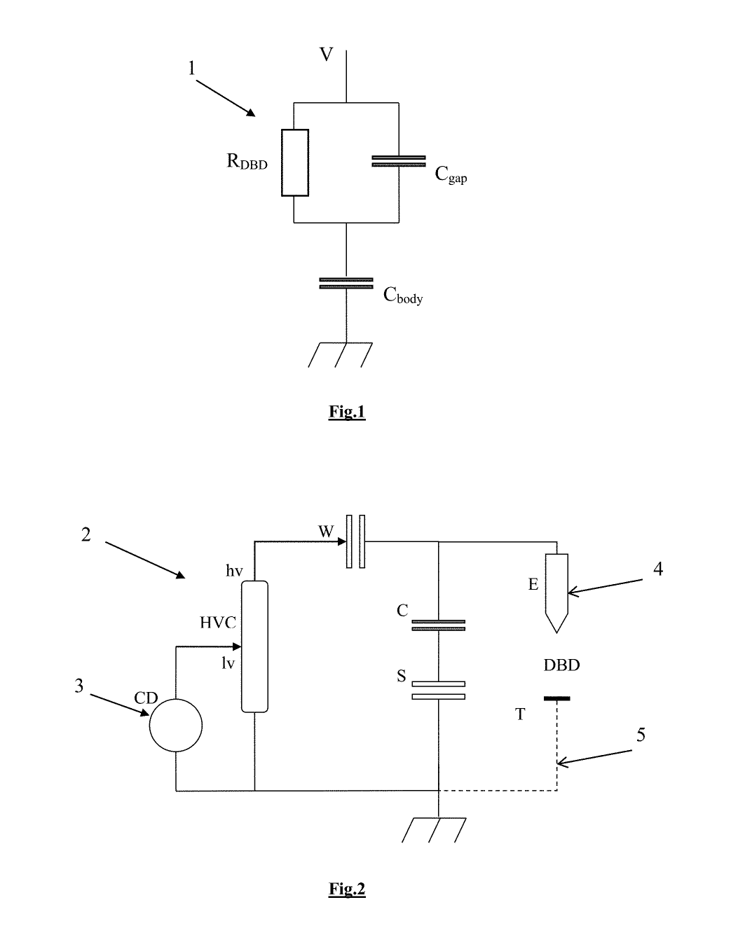

FIG. 1 shows a simplified circuit diagram illustrating the general principles of the invention, where: V represents a pulsed high voltage; C_{gap} is the capacitance of the gap between the electrode and the treated tooth; C_{body} represents the capacitance of the treated body; and R_{DBD} represents the resistance of DBD discharge.

As the human or animal body is predominantly made of water, the tissue dielectric constant is sufficiently high to create DBD between the device electrode and the tooth. Most of the applied voltage appears across the DBD gap between the electrode and the tooth but the electrical current is held at a safe level. Such an arrangement is usually described as a Floating Electrode Dielectric Barrier Discharge (FEDBD), as a ground connection is not necessary. The low temperature, low current and non-destructive nature of the discharge renders this methodology safe for use on patients.

FIG. 2 illustrates an example of a circuit diagram 2, which can be used to generate and apply the DBD. The circuit includes a high voltage ignition coil driver 3, which controls the voltage output from a high voltage coil HVC, between a high voltage terminal hv and a low voltage terminal lv. An electrode 4 and capacitor C are connected in parallel to the high voltage coil though a spark gap W. A second spark gap S is provided between the capacitor and ground. T represents the treated tooth and DBD indicates the location of the dielectric barrier discharge. Grounding of tooth T is not necessary and such an optional connection 5 is shown as a dashed line.

The dielectric covering the electrode 4 may be formed of one or more dielectric coatings. In that regard, the dielectric may be formed of any suitable material such as ceramics, Kapton tape, quartz, glass, polyether ether ketone (PEEK), polypropylene or the like, with a high electric breakdown strength and low dielectric loss.

The device is preferably tunable by adjusting the properties of the driving impulse from the high voltage coil driver 3, such as the amplitude, frequency, duty cycle, waveform, etc. More crudely, the spark gaps, which define the distance between the spark gap electrodes, can be adjusted.

The device could also be tuned by changing the DC power supply voltage of the driver and/or coil driving voltage. Alternatively, other elements of the circuit could be varied such as the electrode, spark gaps, coil or capacitor, which would also affect the behavior of the device.

The device has been described simply for the purpose of illustration and other circuits could instead be used, provided the circuit is capable of generating DBD when the electrode is placed in close proximity to a treated tooth a tooth in need of treatment.

A more advanced version of the device could contain auto-tuning hardware and/or software to optimize the discharge.

Furthermore, a direct (DC) or alternating (AC) bias voltage can be applied using an auxiliary electrode, which is, ideally, electrically separated from the DBD electrode. Such electrode can be touching the tooth directly, or be separated from the tooth. The electrode could be placed remotely from the DBD electrode, for example within the handheld part of the chassis of the device to provide electrical bias or ground connection to the patient's body, or in close proximity to the DBD electrode, for example around the DBD electrode or within the DBD electrode area of the device.

Such auxiliary electrode may be used to limit or expand the treated area, as the electrical field or current created by the auxiliary electrode could inhibit or promote transport of specific ions on the surface of the tooth enamel or within the enamel.

Importantly though, the discharge will always be a pattern of micro-discharges, characteristic of DBD so that there is no spark generation, which might cause discomfort or injury. As such, the invention is non-destructive and safe to use. To protect against spark generation, the electrode is coated in a dielectric material, which ensures the discharge is always DBD.

With the above in mind, the device is operated with short pulses of high voltage and long breaks in between, which means the duty cycle is low compared to a continuous sinusoidal signal. Accordingly, the power required to generate a DBD is minimal, which further renders the device safe for use on patients.

The described circuits are set up to operate with filamental DBD. However, any other similar type of discharge will suffice. For example, a uniform DBD discharge or any other suitable form of atmospheric pressure glow discharge will suffice.

In either case the device, or at least the electrode 4, is preferably portable and hand

As described above, DBD could be created between the device electrode and the treated tooth only when there is a gas, preferably air, present between the electrode and the tooth.

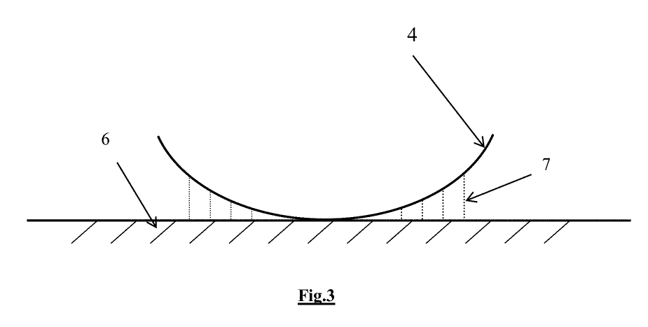

A substantially not flat electrode can be used. FIG. 3 illustrates an example of a curved electrode 4. While electrode 4 may be directly in contact with the surface of the tooth to be treated 6 while a signal is applied from the device, DBD will be created in the air above the treated tooth, indicated by reference numeral 7, at locations adjacent to the contact area between the electrode and the tooth, where the electrode is in close proximity to the tooth.

Moreover, the electrode 4 may be substantially not flat, for example curved, dented, corrugated, pitted, perforated or similar, only locally, creating a number of areas where DBD is present.

Furthermore, the electrode 4 may consist of a number of electrodes.

To apply the DBD to a larger area, or to improve the uniformity of the treatment, a suitably sized and portable electrode 4 could be moved along, in contact or in close proximity to, the surface of the tooth 6.

In particular, the electrode 4 may be of shape of a wire or a thin tape, resembling a dental floss or tape. In one embodiment, such electrode may be made of copper wire 0.1-0.5 mm thick, covered with polyether ether ketone (PEEK). Such electrode might be placed between the teeth to remineralize the enamel on the adjacent interstitial areas of the teeth where tooth decay is frequently initiated.

1. A method for the treatment of teeth comprising:
placing substances on or adjacent to the surface of the tooth enamel;
enhancing the transport of those substances through the tooth enamel by generating a gaseous plasma in proximity to the tooth.

2. The method of claim 1, wherein the treatment comprises remineralization of the tooth.

3. The method of claim 1, wherein the gaseous plasma is a dielectric barrier discharge.

4. The method of claim 1, wherein the method includes:
placing a dielectric coated electrode in proximity to the teeth;
applying a voltage signal to generate a dielectric barrier discharge in air between the teeth and the electrode.

5. The method of claim 1, wherein the method includes:
placing a dielectric coated electrode between adjacent teeth;
applying a voltage signal to generate a dielectric barrier discharge in air between the teeth and the electrode.

6. The method of claim 1, wherein the method includes applying substances containing one or more of fluoride, calcium or phosphate to human or animal teeth in order to remineralize the tooth.
